(19) United States
(12) Patent Application Publication
(10) Pub. No.: US 2004/0249245 A1
(43) Pub. Date: Dec. 9, 2004

(54) Intracorporeal probe for analysis or diagnosis and/or treatment, for example of hollow organs and body cavities in the human or animal body

(76) Inventor: Klaus M. Irion, Liptingen (DE)

Correspondence Address: ST. ONGE STEWARD JOHNSTON & REENS, LLC, 986 BEDFORD STREET, STAMFORD, CT, 06905-5619, US

(21) Appl. No.: 10/800,567

(22) Filed: Mar. 15, 2004

Related U.S. Application Data
(63) Continuation of application No. PCT/EP02/10161, filed on Sep. 11, 2002.

(30) Foreign Application Priority Data
Sep. 14, 2001 (DE) 101 46 197.6

(51) Int. Cl. A61B 1/06

(52) U.S. Cl. 600/160; 600/179

(57) ABSTRACT

An intracorporeal probe for examination and/or treatment, for example of hollow organs or natural or artificially created body cavities in the human or animal body, the probe being in the form of a capsule which can be introduced into the body without external connection elements, has at least one light-emitting element and at least one light-receiving element, the light-receiving element receiving light in another wavelength range than that in which the light-emitting element emits light.

### CROSS-REFERENCE TO RELATED APPLICATIONS

The present application is a continuation of pending international patent application PCT/EP02/10161 filed on Sep. 11, 2002 which designates the United States, and which claims priority of German patent application 101 46 197.6 filed on Sep. 14, 2001.

### BACKGROUND OF THE INVENTION

The invention relates to an intracorporeal probe for examination and/or therapy, for example of hollow organs or natural or artificially created body cavities in the human or animal body, the probe being in the form of a capsule which can be introduced into the body without external connection elements, and the probe having at least one light-emitting element and at least one light-receiving element.

An intracorporeal probe of this kind is known from EP-A-0 667 115.

To examine hollow organs or body cavities, it is known to use imaging methods such as ultrasound, radiography, computed tomography and magnetic resonance tomography. Endoscopic procedures are also known for the same purpose.

Endoscopes are introduced into the body for visual presentation of the inside of the body. Quantitative information, for example information on oxygen content, carbon dioxide content or pH value, is in some cases obtained using probes which are introduced into the body via the instrument channel of the endoscope and are connected to an extracorporeal analysis apparatus.

A more recent field of medical analysis and diagnosis concerns what is called photodynamic diagnosis. In photodynamic diagnosis, photosensitive substances, for example aminolevulinic acid (ALA) or its precursor, are introduced into the tissue to be examined or the tissue region to be examined. Use is made of the fact that photosensitive substances of this kind accumulate in malignant tissue, for example tumors, to a greater extent than they do in healthy tissue. By means of a fluorescence endoscope introduced into the body, the tissue to be examined is irradiated with light, as a result of which the photosensitive substances are excited to fluorescence. The occurrence of fluorescence or the intensity of the fluorescence observed then permits a conclusion to be drawn on whether the examined tissue is healthy or pathologically altered. This method permits visualization of tumors in an early stage.

The disadvantages of current endoscopic systems are that endoscopes cannot be used for examining all areas of the body. For example, areas of the small intestine are not accessible to an endoscope, not even endoscopes which have a flexible shaft. Moreover, endoscopic examinations place a not inconsiderable burden on the investigating physician and also on the patient and they therefore cannot be used for routine examination. The insertion of an endoscopic tube causes considerable discomfort to a patient, for example in gastro-enterology examinations. In addition to this, standard endoscopes do not necessarily provide quantitative data and require image analysis by the investigating physician.

EP-A-0 667 115 mentioned at the outset discloses an intracorporeal probe which is designed in the form of a capsule that can be swallowed by the patient who is to be examined, so as to be able to visually examine the gastrointestinal tract. The optical signals received by the probe are transmitted to outside the body by telemetry via a transmitter present in the capsule and visualized. This known autonomous video probe does indeed make it possible to visually inspect the gastrointestinal tract and transmit the images by telemetry to outside the body, but analysis of the transmitted image has to be made by the experienced physician. The physician must evaluate and assess the image data throughout the passage of the video probe through the gastrointestinal tract, which can take over eight hours. Because of the arbitrary position in the hollow organ, a large number of the images do not provide any information capable of being evaluated. Moreover, a disadvantage of this known video probe is that the image quality can be reduced by mucus and such like soiling the surface of the lens.

### SUMMARY OF THE INVENTION

It is therefore an object of the invention to take an intracorporeal probe of the type mentioned at the outset and develop it in such a way that it permits specific detection of diagnostically relevant visual information without the need for elaborate image analysis.

According to the invention, an intracorporeal probe for at least one of examination and therapy of body cavities in the human or animal body is provided, the probe being in the form of a capsule which can be introduced into the body without external connection elements, and the probe having at least one light-emitting element and at least one light-receiving element, wherein the at least one light-receiving element receives light in another wavelength range than that in which the at least one light-emitting element emits light.

With the intracorporeal probe according to the invention, it is possible to detect diagnostically relevant information visually, but without the need for optical imaging. By virtue of the fact that the at least one light-receiving element receives light in another wavelength range than that in which the at least one light-emitting element emits light, it is possible to use the light of the light-emitting element to excite tissuespecific photosensitizers in the same way as in photodynamic diagnosis, i.e. when the light-emitting element emits an excitation wavelength, and to determine its fluorescence by means of the at least one light-receiving element receiving the emission wavelength of the fluorescence. This permits characterization of the state of the tissue, without the tissue having to be visually imaged for this purpose. In the simplest case, the information obtained via the intracorporeal probe according to the invention, which information is preferably transmitted to outside the body by telemetry, concerns whether a fluorescence was detected (malignant tissue) or not detected (healthy tissue). Problems of blurred image transmission, of the kind that can arise when the lens of the intracorporeal probe is soiled, as happens in the known intracorporeal probe, do not arise. The treating physician does not have to analyze or interpret a visual image for a pathological condition, and instead simply the presence of a signal (fluorescence present or absent) affords the physician a finding. Endogenous substances (for example fluorophores) which provide auto-fluorescence can be used as photosensitizers, or external photosensitizers are administered which are tissue-specific and emit fluorescence. To determine the state of the organ, it is also possible to administer pure fluorescent substances which are not incorporated in a tissue-specific manner, for example in blood. Such substances, for example sodium fluorescein, can be used for detection of bleeding, for example in the stomach or in the intestine, and can be administered by injection. With the intracorporeal probe according to the invention, an autonomous probe is made available which is suitable in particular for photodynamic diagnosis and which has a higher level of acceptance compared to the known endoscope systems, which sometimes cause the patient some inconvenience.

The described probe can also remain as an implant in the body. This permits, for example, intracorporeal monitoring of therapy in the postoperative stage.

In a preferred embodiment, the light of the at least one light-emitting element has a shorter wavelength than the light which can be received by the at least one light-receiving element.

In this embodiment, the intracorporeal probe according to the invention is advantageously suitable in particular for use in the context of photodynamic diagnosis, because the light emitted at a shorter wavelength can be used to excite endogenous or administered photosensitive substances, and the fluorescent light can then be received by the light-receiving element, the received light being clearly separate from the emitted light in spectral terms, so that no erroneous interpretations of the data delivered by the probe are possible.

In a further preferred embodiment, the at least one light-emitting element has an emission characteristic covering the entire solid angle and/or a plurality of light-emitting elements are arranged in the capsule in such a way that the light emission covers the entire solid angle.

The aforementioned measures, which can be provided separately or in combination with one another in the intracorporeal probe according to the invention, have the advantage that the emission of the light-emitting element or elements takes place uniformly in all spatial directions, largely independently of the position of the probe in the hollow organ or in the body cavity.

In a further preferred embodiment, the at least one light-emitting element is a light-emitting diode (LED).

The advantage of this is that, by using light-emitting diodes as light-emitting elements, the intracorporeal probe can be produced at low cost. Moreover, light-emitting diodes have the advantage of a high luminous efficiency.

In a further preferred embodiment, the light-emitting diode emits in the blue frequency range.

A light-emitting diode emitting in the blue frequency range is particularly suitable for excitation of fluorescence and, consequently, for use of the intracorporeal probe according to the invention for fluorescence diagnosis of tissue in the human or animal body.

In a further preferred embodiment, the at least one light-receiving element is designed in such a way that it receives light from the entire solid angle range and/or a plurality of light-receiving elements are arranged in the capsule in such a way that light can be received from the entire solid angle range.

With these aforementioned measures, which can be used separately or in combination with one another in the probe according to the invention, the advantage once again is that the light coming from the examination tissue can be received uniformly by the probe, largely independently of the position of the probe in the body.

In a further preferred embodiment, an optical filter element is arranged on the at least one light-emitting element and/or on the at least one light-receiving element.

This measure has the advantage that a particularly good separation of the wavelength ranges between the emitted light and the received light is achieved, with the result in particular that the at least one light-receiving element only receives the light relevant for the diagnosis. The optical filters used can in particular be interference filters placed in front of the light-emitting and light-receiving elements.

In a further preferred embodiment, the capsule contains at least one light-receiving element in the form of an image sensor, in particular of a two-dimensional image sensor, for the purpose of receiving a visual image.

The advantage of this is that, in addition to the spectral information gained using the at least one light-receiving element, imaging information can also be obtained which permits visualization of the examined hollow organ or the examined body cavity. However, as has already been mentioned, the main focus of the present invention lies not in visual representation, but in obtaining optical data which do not require any visual representation of the examined tissue area. Visual representation of the examined tissue area does, however, have the added advantage of easier orientation for the physician.

Correspondingly, in a further preferred embodiment, the capsule contains at least one light-emitting element which emits white light.

The advantage of this is that illumination of the body cavity or hollow organ with white light permits a more true to nature visualization of the examined tissue area than is obtained by illumination with colored light.

In a further preferred embodiment, the capsule contains a transmitter element for the purpose of emitting signals from the probe to outside the body.

The advantage of this is that the optical information received by the probe and optionally prepared by a signal-processing unit inside the capsule can be made available instantaneously to the treating physician via a corresponding receiving unit and an associated further display unit, while the probe can remain in the body.

It is further preferred for the capsule to contain a signal-preprocessing element which forwards the opto-electrical signal originating from the at least one light-receiving element to the transmitter element.

A signal-preprocessing element of this kind has the advantage of permitting preprocessing of the light signals received by the at least one light-receiving element in such a way that only the signals relevant for the diagnosis are transmitted to the physician. Moreover, in pulsed operation of the at least one light-emitting element, the signal-preprocessing element can perform synchronization between the pulsed emission and the reception of light by the at least one light-receiving element.

In a further preferred embodiment, the capsule contains a signal storage element for the purpose of storing signals of the at least one light-receiving element.

This measure is particularly of advantage if the light signals received by the at least one light-receiving element are not to be immediately processed or are not to be transmitted immediately to outside the body by telemetry, and instead the data obtained by the probe are to be evaluated at a later time. This in particular has the advantage that a number of patients with an intracorporeal probe according to the invention can be diagnosed simultaneously, and the individual intracorporeal probes and their data can then be read out and evaluated after removal of the probe from the body.

In a further preferred embodiment, the capsule contains a position-detecting element whose position can be determined from outside the body.

This measure has the advantage that the position of the probe inside the patient's body can be monitored. This is particularly of advantage if the probe is located in a body cavity of the patient where, because of natural peristalsis, it does not lie fixed in the body but is instead moved. Since the spatial position of the probe about its probe axis is also changed during such movement, position determination also permits constant monitoring of the position of the probe in relation to its own axis and thus also of the position relative to the tissue to be examined.

In this respect it is also preferred if the position-detecting element is designed as a coil system whose position can be detected via an external magnetic field detector.

A coil system of this kind used as position-detecting element can advantageously be of a miniaturized design, so that it is particularly advantageously suitable for design of a miniaturized intracorporeal probe.

Locating of the probe in the body is necessary especially when the probe shows a positive analysis result or diagnosis result. In this case, provision can be made to electrically activate magnetic coil systems in order to determine the position of the probe in the body. Of course, such a coil system requires that the outer wall of the capsule or the shell of the capsule is not made of metal, and also that as little metal as possible is used inside the capsule.

In a further preferred embodiment, the capsule contains a positioning element which can be controlled from outside the body in order to position the probe.

This measure has the advantage that the probe can be fixed at a desired location and in a defined position inside the body cavity or hollow organ. This is of advantage particularly in body cavities or body organs which have a much greater cross section compared to the size of the probe, so that, without such a positioning measure, it would not be possible to fix the probe at a predetermined location and in a defined position. Particularly in organs with peristalsis, this measure has the advantage of more targeted and more exact diagnosis.

In a further preferred embodiment, the capsule contains an energy supply unit or an element for receiving electromagnetic energy irradiated from outside the body.

The advantage of this is that the intracorporeal probe according to the invention is autonomous in respect of its energy supply, i.e. it does not require any external connection lines for supply of energy. The energy supply element can, for example, be a miniaturized battery or an energy storage element with energy converter which converts into electrical energy.

In a further preferred embodiment, fluorescent/luminescent marker substances are arranged on the capsule.

This measure has the advantage that the marker substances can interact with their environment, for example blood, so that parameters such as oxygen content, carbon dioxide content, etc., can be deduced.

The capsule can also preferably contain a luminescent substance which is excited to luminescence from outside the body, for example by electromagnetic energy, and emits light through the transparent capsule shell. This excitation can also be effected outside the body by X-radiation, by using a dye sensitive to X-rays, or the excitation can be effected outside the body by ultrasound energy, by using photoacoustic dyes. Examples of dyes that can generally be used are sodium fluorescein (phthalem), eosin, rhodamine and derivatives thereof. Such a luminescent substance inside the capsule then serves as the at least one light-emitting element which does not require any energy supply present in the capsule.

In a further preferred embodiment, the capsule contains a reservoir for therapeutic substances and/or diagnostic substances which are dispensed inside the body by the probe.

The advantage of this is that the aforementioned substances can be introduced into the body together with the probe, so that no additional treatment step is needed for administering these substances, and, what is more, the substances can be brought more exactly to the desired site where the examination also takes place by means of the probe.

To treat malignant or pre-malignant changes, it is possible to administer a photosensitizer with a photodynamic/therapeutic action which is triggered by irradiation with light. Examples of other photosensitive substances suitable for diagnosis and/or therapy are porphyrins (protoporphyrin IX, for example induced by aminolevulinic acid (ALA), benzoporphyrin), metatetra(hydroxyphenyl)chlorin (m-THPC), cyanines (phthalocyanines (Zn-phthalocyanine)), hypericin, tin ethyl etiopurpurin (SnET₂), lutetium texaphyrin and their derivatives, or others.

In a further preferred embodiment, an ultrasound transmitter/receiver element for ultrasound imaging is arranged in the capsule.

The advantage of this is that ultrasound imaging permits sectional imaging of deeper-lying tissue areas which, because of the lower depth of penetration of light in tissue, cannot be determined or can be determined only inadequately by optical means.

In a further preferred embodiment, the probe has at least one line leading to outside the body for the purpose of exchanging information, energy and/or substances.

In a further preferred embodiment, the probe is designed as an implant, and a capsule wall is formed with long-term biocompatible and sterilizable material.

The advantage of this is that the probe can also be designed as a long-term implant, for example for implantation in an already resected tumor bed, because the capsule wall is made biocompatible and sterilizable. For use of the probe as a long-term implant, the probe should additionally have a stable design. An example of a possible use of such a probe implant is in the treatment of (severe) glioblastoma in neurosurgery. In this critical pathological condition, it is in most cases impossible to resect and excise all the tumor components and deeper-lying tumor cells by means of surgery. With a design of the probe according to the invention as a long-term implant, it is also possible, following surgery, to continue treatment inside the body.

In a further preferred embodiment, the probe, as an implant, has a fully enclosing transparent capsule wall, preferably made of glass.

Designing the entire capsule wall as a stable glass wall has the advantage of affording transparency and biocompatibility per se. By means of possible integration of light-scattering components, it is possible to further improve the homogeneity of the emission through the capsule wall. Glass as the material for the capsule wall also advantageously provides the integrated components with a certain level of protection against high-energy (“hard”) radiation which is often used for continued postoperative treatment, so that the probe is suitable as a long-term implant in connection with this aspect too.

As has already been mentioned, the intracorporeal probe according to the invention is suitable not only for diagnostic purposes, but also for therapeutic purposes, as has been described in one of the aforementioned embodiments.

To this end, in a further preferred embodiment the capsule contains at least one element or element array emitting therapeutic light, which element or element array emits light for photodynamic therapy principally in the wavelength range in which the absorption peak of a photosensitizer introduced into the body lies.

This measure has the advantage that, by integration of elements which emit therapeutic light, in particular those whose wavelength lies in the absorption peak of the photosensitizer, an optimal effect of the photodynamic therapy can be achieved.

In a further preferred embodiment, the at least one element or element array emitting therapeutic light is arranged in the capsule in such a way that the whole solid angle is illuminated homogeneously.

The advantage of this is that the whole solid angle of the body area to be treated can be irradiated homogeneously with therapeutic light.

In a further preferred embodiment, the at least one light-emitting element, the at least one light-receiving element and the at least one element emitting therapeutic light are oriented in such a way that therapeutic light can be emitted locally in the solid angle in which a fluorescence signal is received by the at least one light-receiving element, which signal is produced by the exciting light emitted by the at least one light-emitting element.

This measure has the advantage that only tissue areas detected beforehand as being malignant areas, i.e. fluorescent areas, on the basis of the diagnosis by the light-emitting and light-receiving elements, are irradiated with therapeutic light in said solid angle, i.e. this arrangement permits locally directed therapy.

In a further preferred embodiment, the at least one element emitting therapeutic light is designed as a light-emitting diode with a wavelength in the range of 590 to 650 nm.

This measure has the advantage that the absorption peaks of many photosensitizers, such as aminolevuliriic acid (ALA), hypericin etc., lie in this wavelength range, and that the power of the light-emitting diodes is relatively high compared to light-emitting diodes which emit at a short range.

Further advantages or features will become evident from the following description and from the attached drawing. It will be appreciated that the aforementioned features and those still to be explained below can be used not only in the respectively stated combination, but also in other combinations or in isolation, without departing from the scope of the present invention.

### BRIEF DESCRIPTION OF THE DRAWINGS

An illustrative embodiment of the invention is shown in the drawing and is described in more detail below with reference to this drawing, in which:

FIG. 1 shows a diagrammatic representation of an intracorporeal probe in a side view and on a greatly enlarged scale;

FIG. 2 shows a section through the intracorporeal probe from FIG. 1, along line II-II in FIG. 1; and

FIG. 3 shows a diagrammatic representation in which the intracorporeal probe from FIG. 1 is present in a hollow organ of a human body.

### DETAILED DESCRIPTION OF PREFERRED EMBODIMENTS

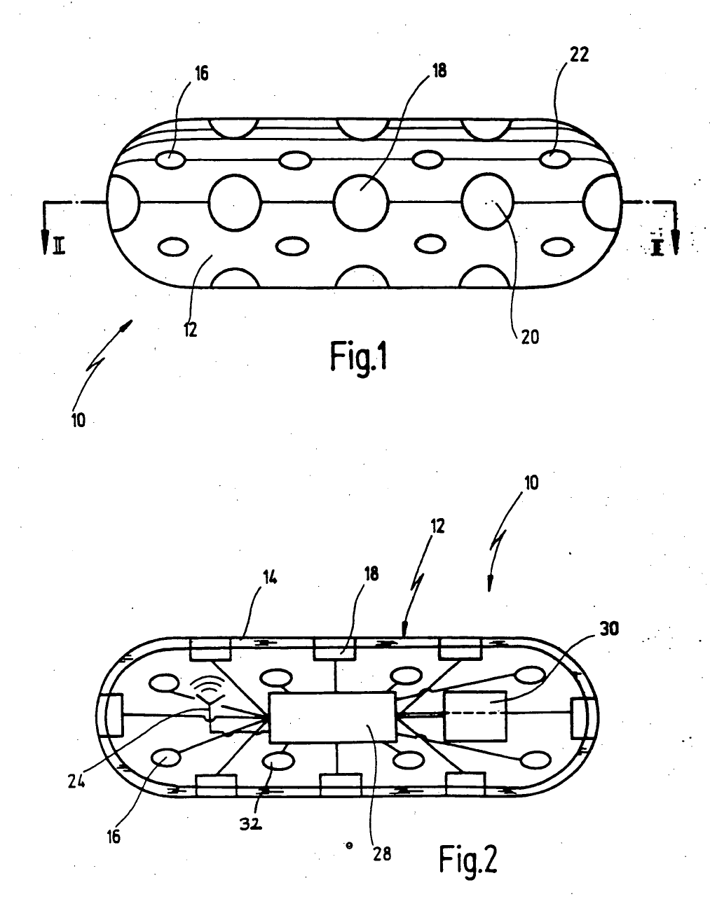

In FIGS. 1 and 2, an intracorporeal probe has been provided with general reference number 10 and is used, for example, to examine hollow organs or body cavities in the human or animal body. The probe 10 serves in particular as an autonomous probe for use in photodynamic diagnosis. The capsule 12 has a preferably transparent capsule shell or capsule wall 14.

A plurality of light-emitting elements 16 designed as light-emitting diodes are arranged in the capsule. The light-emitting elements 16 are distributed in the capsule in such a way that the light emission covers the entire solid angle, i.e. the probe 10 emits light in all spatial directions, and as uniformly as possible.

The light-emitting elements 16 designed as light-emitting diodes emit light in the blue frequency range, for example at a wavelength of approximately 480 nm, when sodium fluorescein is used.

Moreover, the capsule 12 contains light-receiving elements 18 in the form of photoelectric elements for receiving light. The light-receiving elements 18 are able to receive light in another wavelength range than that in which the light-emitting elements emit light. The spectral properties of the light-receiving elements 18 are accordingly separate from the spectral properties of the light-emitting elements 16. In the present illustrative embodiment, the sensitivity of the light-receiving elements 18 starts at a wavelength of >500 nm when sodium fluorescein is used.

The light sensitivity of the light-receiving elements 18 is in a wavelength range with a longer wavelength than the light emitted by the light-emitting elements 16.

For still better separation of the spectral ranges, optical filter elements, for example in the form of interference filters, can be arranged in front of the light-emitting elements 16 and/or in front of the light-receiving elements 18.

The light-receiving elements 18 are distributed in the capsule in such a way that they can receive light from the entire solid angle range.

One or more of the light-receiving elements 18 can also be designed in the form of an image sensor, in particular a two-dimensional image sensor, for receiving a visual image, as is illustrated by way of example for a light-receiving element 20 in FIG. 1.

The light-receiving element 20 can for example be a CCD sensor or a CMOS sensor.

Correspondingly, one of the light-emitting elements 16 can also be designed as a white light source, as is illustrated by way of example for one light-emitting element 22, in order to illuminate the observation area in the human or animal body in a manner as true to nature as possible. The element 22 is preferably a diode emitting white light.

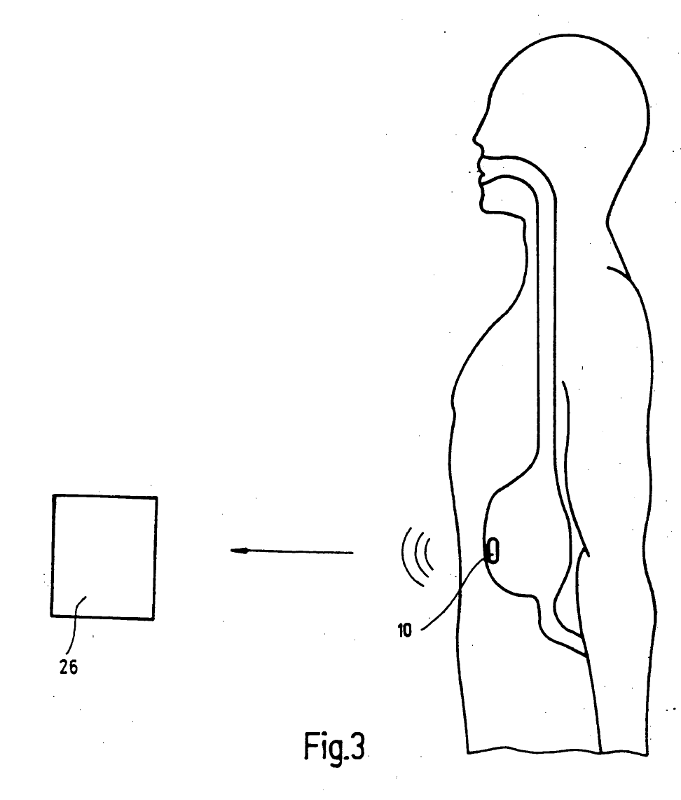

The capsule 12 also contains a transmitter element 24 which emits signals from the probe to outside the body, as is shown diagrammatically in FIG. 3. The signals emitted from the probe 10 are then received by an extracorporeal receiver 26 and presented to the treating physician, for example on a display unit or a screen.

According to FIG. 2, a signal-preprocessing element 28 is like-wise arranged in the capsule 12. The signal-preprocessing element 28 takes the opto-electrical signals coming from the light-receiving elements 18 and forwards these signals to the transmitter element 24. Correspondingly, all the light-receiving elements 18 are connected electrically to the signal-preprocessing element 28. The light-emitting elements 16 are also all connected to the signal-preprocessing element 28.

Thus, for example, in pulsed operation of the light-emitting elements 16, the signal-preprocessing element can also perform synchronization between the pulsed light emission and the reception of the signals of the light-receiving elements 18.

As an alternative or in addition to the transmitter element 24, the capsule can contain a signal storage element (not shown) for storing signals of the light-receiving elements 18.

The capsule 12 also contains an energy supply element 30 which supplies energy to the light-emitting elements 16 and light-receiving elements 18, and which is connected to the signal-preprocessing element 28. The energy supply element 30 is for example a micro-battery, although it can also be an element for receiving electromagnetic energy irradiated from outside the body.

To be able to monitor the position of the probe 10 in the human body after it has been introduced, the capsule contains a position-detecting element (not shown) whose position can be determined from outside the body. A position-detecting element of this kind is preferably designed as a coil system whose position can be detected via an external magnetic field detector. It will be appreciated that the capsule wall 14 is correspondingly designed such that it does not have a magnetic shielding action.

In FIG. 3, the probe 10 is shown positioned in the stomach of a patient. To fix the probe 10 in the position shown, the capsule also preferably contains a positioning element which can be controlled from outside the body for the purpose of positioning the probe. Such positioning can, for example, be done from outside the body by the action of a magnetic field.

The capsule can also contain a reservoir for therapeutic substances and/or diagnostic substances, and these substances can then be gradually dispensed inside the body by the probe, for example through a membrane (not shown) in the capsule wall 14.

Finally, an ultrasound transmitter/receiver element for ultrasound imaging can be arranged in the probe 10.

Fluorescent or luminescent marker substances are preferably applied on the outside of the capsule 12, it being possible to use sodium fluorescein as one such dye. Bleeding can be detected with a dye of this kind.

The capsule 12 can also contain luminescent substances which can be made to illuminate by excitation from outside the body, for example via electromagnetic energy, X-radiation or ultrasound energy, and can thus serve for light emission.

In an application of the intracorporeal probe 10 for photodynamic diagnosis, a photosensitizer, which can be an endogenous substance or a substance administered from outside and settling in a tissue-specific manner, is excited to fluorescence by means of the light-emitting elements 16, and the fluorescent light is then received by means of the light-receiving elements 18. To assess whether the examined tissue is healthy or pathologically altered, it suffices for the probe 10 to transmit, to outside the body, a signal which contains the information.

“Fluorescence present” (pathologically altered tissue) or “Fluorescence not present” (healthy tissue).

A finding is thus made available to the physician without the investigated tissue being visually imaged.

The probe 10 can also be designed as an implant, in which case the capsule wall 14 is then designed with long-term biocompatible and sterilizable material. The capsule wall 14 of the probe 10 is in particular transparent and is preferably made of glass, in which case light-scattering particles can be integrated into the glass.

The capsule 12 can also contain at least one element 32 which emits therapeutic light for photodynamic therapy, principally in the wavelength range in which the absorption peak of a photosensitizer previously introduced into the patient's body lies.

Concerning the at least one element 32 which emits therapeutic light and can also be designed as an element array, provision is also made for the entire solid angle to be illuminated homogeneously with therapeutic light. In this respect it is advantageous if the at least one element 32 emitting therapeutic light, the at least one light-emitting element 16 and the at least one light-receiving element 18 are oriented in such a way that the therapeutic light from the at least one element 32 emitting therapeutic light is emitted in a solid angle in which a fluorescence signal was received beforehand by the at least one light-receiving element 18, which signal is the result of excitation of the light emitted by the at least one light-emitting element 16.

The at least one element emitting therapeutic light is preferably a light-emitting diode with an emission wavelength in the range of 590 to 650 nm.

Applications of the intracorporeal probe 10 can include examination of the gastrointestinal tract for malignant changes with administration of a photosensitizer which accumulates in a tumor-specific manner, recording of bleeding in the stomach, colon and above all in the small intestine, after surgery involving intravenous administration of a fluorescence dye, photodynamic therapy of malignant or pre-malignant tissue by light exposure and administration of a photodynamic/therapeutic photosensitizer, or treatment monitoring (for example detection of bleeding) after open surgery and noninvasive surgery, to name but a few examples.

What is claimed is:

1. An intracorporeal probe for at least one of examination and therapy of body cavities in the human or animal body, said probe being in the form of a capsule which can be introduced into the body without external connection elements, and said probe having at least one light-emitting element and at least one light-receiving element, wherein said at least one light-receiving element receives light in another wavelength range than that in which said at least one light-emitting element emits light.

2. The probe of claim 1, wherein said light of said at least one light-emitting element has a shorter wavelength than said light which can be received by said at least one light-receiving element.

3. The probe of claim 1, wherein said at least one light-emitting element has an emission characteristic covering the entire solid angle.

4. The probe of claim 1, wherein a plurality of light-emitting elements are arranged in said capsule in such a way that the light emission covers the entire solid angle.

5. The probe of claim 1, wherein said at least one light-emitting element is a light-emitting diode.

6. The probe of claim 5, wherein said light-emitting diode emits in the blue frequency range.

7. The probe of claim 1, wherein said at least one light-receiving element is designed in such a way that it receives light from the entire solid angle range.

8. The probe of claim 1, wherein a plurality of light-receiving elements are arranged in said capsule in such a way that light can be received from the entire solid angle range.

9. The probe of claim 1, wherein an optical filter element is arranged on at least one of said at least one light-emitting element and said at least one light-receiving element.

10. The probe of claim 1, wherein said capsule contains at least one further light-receiving element in the form of an image sensor for the purpose of receiving a visual image.

11. The probe of claim 10, wherein said capsule contains at least one further light-emitting element which emits white light.

12. The probe of claim 1, wherein said capsule contains a transmitter element for the purpose of emitting signals from said probe to outside the body.

13. The probe of claim 12, wherein said capsule contains a signal-preprocessing element which forwards an opto-electrical signal originating from said at least one light-receiving element to said transmitter element.

14. The probe of claim 1, wherein said capsule contains a signal storage element for the purpose of storing signals of said at least one light-receiving element.

15. The probe of claim 1, wherein said capsule contains a position-detecting element whose position can be determined from outside the body.

16. The probe of claim 15, wherein said position-detecting element is designed as a coil system whose position can be detected via an external magnetic field detector.

17. The probe of claim 1, wherein said capsule contains a positioning element which can be controlled from outside the body in order to position said probe.

18. The probe of claim 1, wherein said capsule contains at least one of an energy supply unit and an element for receiving electromagnetic energy irradiated from outside the body.

19. The probe of claim 1, wherein fluorescent/luminescent marker substances are arranged on said capsule.

20. The probe of claim 1, wherein said capsule contains at least one luminescent substance which can be excited by excitation from outside the body and emits light through a capsule wall.

21. The probe of claim 1, wherein said capsule contains a reservoir for at least one of therapeutic substances and diagnostic substances which are dispensed inside the body by said probe.

22. The probe of claim 1, wherein an ultrasound transmitter/receiver element for ultrasound imaging is arranged in said probe.

23. The probe of claim 1, wherein said probe has at least one line leading to outside the body for the purpose of exchanging at least one of information, energy and substances.

24. The probe of claim 1, wherein said probe is designed as an implant and has a capsule wall formed with long-term biocompatible and sterilizable material.

25. The probe of claim 1, wherein said probe has a fully enclosing transparent capsule wall.

26. The probe of claim 1, wherein said capsule contains at least one element emitting therapeutic light, said element emitting light for photodynamic therapy principally in the wavelength range in which an absorption peak of a photosensitizer introduced into the body lies.

27. The probe of claim 26, wherein said at least one element emitting therapeutic light is arranged in said capsule in such a way that the whole solid angle is illuminated homogenously.

28. The probe of claim 1, wherein said capsule contains at least one element emitting therapeutic light, said element emitting light for photodynamic therapy principally in the wavelength range in which an absorption peak of a photosenstizer introduced into the body lies, and wherein said at least one light-receiving element and said at least one element emitting therapeutic light are oriented in such a way that therapeutic light can be emitted locally in the solid angle in which a fluorescent signal is received by said at least one light-receiving element, which signal is produced by excitation light emitted by said at least one light-emitting element.

29. The probe of claim 1, wherein said capsule contains at least one element emitting therapeutic light, said element emitting light for photodynamic therapy principally in the wavelength range in which an absorption peak of a photosensitizer introduced into the body lies, and wherein said at least one element emitting therapeutic light is designed as a light-emitting diode with a wavelength in the range of about 590 to about 650 nm.

30. The probe of claim 1, wherein said capsule contains a position-detecting element whose position can be determined from outside the body and which is capable to detect at least one of a position and an orientation of said probe with respect to axes of said probe relative to said body cavity.
